(19) United States
(12) Patent Application Publication
(10) Pub. No.: US 2021/0222154 A1
(43) Pub. Date: Jul. 22, 2021

(54) Operation method of magnetic particles

(71) Applicant: SHIMADZU CORPORATION, Kyoto-shi (JP)

(72) Inventors: Akira MURAMATSU, Kyoto-shi (JP); Masamitsu SHIKATA, Kyoto-shi (JP); Masaki KANAI, Kyoto-shi (JP); Seiya FUJIWARA, Kyoto-shi (JP); Tetsuo OHASHI, Kyoto-shi (JP)

(73) Assignee: SHIMADZU CORPORATION, Kyoto-shi (JP)

(21) Appl. No.: 17/154,525

(22) Filed: Jan. 21, 2021

(30) Foreign Application Priority Data
Jan. 22, 2020 (JP) 2020-008544

(51) Int. Cl. C12N 15/10; G01N 1/40; C12Q 1/6806

(52) U.S. Cl. CPC C12N 15/1013; C12Q 1/6806; G01N 1/405

(57) ABSTRACT

The present invention relates to a method for operating magnetic particles in a device in which gel-like medium layer and liquid layer are alternately arranged inside the hollow tube and, first liquid layer and second liquid layer are separated by gel-like medium layer, and magnetic solid with large particle size exists in the first liquid layer rather than magnetic particles and the said magnetic particles. The method of the present invention comprises the first step of moving the magnetic particles and the magnetic solid in the first liquid layer to the gel-like medium layer, the second step of moving the magnetic particles and the magnetic solid moved into the gel-like medium layer into the second liquid layer, and the third step of inverting the movement of the magnetic particles and the magnetic solid moved into the second liquid layer into the gel-like medium layer, by operating a magnetic field.

### TECHNICAL FIELD

The present invention relates to an operation method of magnetic particles for performing separation, extraction, purification, elution and analysis or the like of a target substance which is a biological component such as nucleic acid in a hollow tube in which gel-like medium layers and liquid layers are alternately arranged.

### BACKGROUND TECHNOLOGY

In medical inspection of biological samples, inspection for food safety and health management and inspection of environmental samples for environmental monitoring, etc., the target substance is required to be extracted, separated and purified from a sample containing a wide variety of impurities and subjected to a detection process or a measurement reaction. For example, in genetic testing, nucleic acid (DNA or RNA) must be efficiently separated and purified from biological sample, such as animal and plant blood, serum, cells, urine, feces, etc. and viruses, to remove impurities that affect PCR (Polymerase Chain Reaction) before being subjected to a detection process that involves amplification by PCR to detect the target nucleic acid.

A method of using magnetic particles capable of specifically adsorbing a target substance in order to separate and purify the target substance in a sample has been conventionally known. In this method, water-insoluble magnetic particles having a particle size of about 0.5 μm to a dozen μm, which has a chemical affinity with a target substance such as nucleic acid and a molecular recognition function on the surface of the particles, are used. The magnetic particles are first brought into contact with a biological sample containing target substance, which is dissolved biological sample, and target substance is fixed on the particle surface, and then the magnetic particles are separated and recovered from the liquid phase by magnetic field operation. The recovered magnetic particles are redispersed in a liquid phase such as a cleaning liquid as needed, and the steps of separating and recovering the magnetic particles from the liquid phase are repeated. In this step, impurities non-specifically adsorbed on the particle surface are removed by a washing operation. Then, by dispersing the magnetic particles in the eluate, the target substance fixed to the magnetic particles is released into the eluate, and the target substance in the eluate is recovered. In this method, since the magnetic particles can be separated from the liquid phase by magnetic field operation using a magnet or the like, solid-liquid separation by centrifugal operation becomes unnecessary, which is advantageous for automation of separation and purification of the target substance.

For example, a device has also been developed wherein recovery of magnetic particles from in the liquid phase and disperses of magnetic particles into the liquid phase are performed by pipette operation, and the entire series of operations from adsorption of target substance in the sample to magnetic particles to separation and purification of cleaning and elution are performed by automatic machine (For example, Patent Document 1). On the other hand, instead of pipette operation, a method has been proposed in which a target substance is separated and purified by moving magnetic particles along the long axis direction of a tube in this device using tubular device in which water-based liquid layer such as dissolution/fixative, cleaning solution, eluate, etc. and water-insoluble gel-like medium layer are alternately layered (For example, Patent Document 2). This tubular device is configured such that magnetic particles dispersed in an aqueous liquid layer move by a magnetic field operation from the outside of a capillary tube and pass through a water-insoluble gel-like medium layer. Therefore, in the method using this tubular device, a series of operations can be performed in the same container while maintaining a closed state, so that the risk of contamination is reduced as compared with the pipette operation performed in an open system.

When the target substance in the sample is separated and purified by using the above-mentioned tubular device in which water-based liquid layer and water-insoluble gel-like medium layer are alternately layered, the magnetic particles aggregated by the magnetic force may aggregate with each other. When the magnetic particles are aggregated, the contaminants taken into the gaps between the aggregated particles are not sufficiently washed by the cleaning liquid, and the purification of the target substance becomes insufficient, alternatively, the target substance incorporated in the gaps between the agglomerated particles may not be sufficiently exposed to the eluate, resulting in problems such as insufficient recovery of the target substance. Also, if the target substance can be fixed to the magnetic particles with high efficiency, pretreatment such as removing impurities in the sample in advance becomes unnecessary, and the target substance in the sample can be separated and purified more easily. To make these things possible, in the aqueous liquid layer, a method for operating magnetic particles has been proposed in which magnetic field operation is performed in a state where magnetic particles and a magnetic solid having a particle size larger than that of the magnetic particles coexist (Patent Documents 3 and 4). According to these methods, it has been reported that magnetic particles are efficiently dispersed in a liquid as magnetic solid moves due to magnetic force, target substance can be efficiently fixed to the surface of magnetic particles, and the cleaning efficiency and recovery rate of target substance are improved.

### PRIOR ART DOCUMENT

### Patent Document

[Patent Document 1] WO97/44671
[Patent Document 2] WO2012/086243
[Patent Document 3] WO2015/136689
[Patent Document 4] WO2015/177933

### SUMMARY OF INVENTION

### Problems to be Solved by the Invention

As described above, when separating and purifying target substance in the sample using tubular device in which water-based liquid layer and water-insoluble gel-like medium layer are alternately layered, magnetic particles with fixed target substance is washed with water-based liquid layer composed of a cleaning solution to remove contamination, and then it is transferred by magnetic field operation to the water-insoluble gel-like medium layer together with magnetic solid. Next, the magnetic particles and the magnetic solid are moved from the water-insoluble gel-like medium layer to the aqueous liquid layer composed of the eluate of the target substance by magnetic force operation. Since the aqueous liquid layer composed of the eluate contains the target substance liberated from the magnetic particles, the target substance is collected together with the aqueous liquid (eluate) and subjected to the next detection process or measurement reaction. However, since the magnetic material particles still exist in the aqueous liquid (eluent) in a dispersed state, the target substance is collected in a state in which the magnetic material particles are mixed.

In order to remove the magnetic particles from the aqueous liquid (eluent) containing the target substance, a step of removing the mixed magnetic particles by centrifuging the collected eluate is required, which is complicated for the operator. Also, if the eluate contains magnetic particles and is subsequently subjected to detection process or measurement reaction, when the target substance is nucleic acid (DNA or RNA), magnetic particles may block the optical path in the optical measurement of fluorescence, etc. in PCR, affecting measurement accuracy and sensitivity.

The present invention has been made in consideration of the above circumstances. In the present invention, it is not necessary to separately remove the magnetic particles from the water-based liquid (eluent) containing the purified target substance by centrifugation or the like, and when the target substance is nucleic acid (DNA or RNA), it is an object of the present invention to provide a method for operating magnetic particles, which makes it possible to collect an eluate that can be immediately used for PCR.

### Means for Solving Problems

That is, the object of the present invention is achieved by the following invention.
[1]

An operation method of magnetic particles, in a device wherein a gel-like medium layer and a liquid layer are alternately arranged in a hollow tube having an open end which may be closed on one side and a closed end on the other side, and the first liquid layer and second liquid layer are separated by a gel-like medium layer, and a magnetic particle and a magnetic solid having a particle size larger than that of the magnetic particle are present in the first liquid layer;

which comprises

the first step of moving the magnetic particles and the magnetic solid in the first liquid layer to the gel-like medium layer by operating a magnetic field from the outside of the hollow tube;

the second step of moving the magnetic particles and the magnetic solid which were moved into the gel-like medium layer into the second liquid layer by operating a magnetic field from the outside of the hollow tube; and,

the third step of reversing and moving the magnetic particles and the magnetic solid which were moved into the second liquid layer into the gel-like medium layer by operating a magnetic field from the outside of the hollow tube.
[2]

The operation method according to [1], wherein the magnetic solid has a particle size of 100 nm or more.
[3]

The operation method according to [1] or [2], wherein the particle size of the magnetic solid is 10 times or more the particle size of the magnetic particles.
[4]

The operation method according to any one of [1] to [3], wherein the magnetic solid has a protective layer on the surface of the magnetic solid to prevent corrosion in the liquid layer.
[5]

The operation method according to any one of [1] to [4], wherein the magnetic particles are particles capable of selectively fixing a target substance in a sample which is introduced into the hollow tube to the particle surface.
[6]

The operation method according to [5], wherein the target substance is at least one selected from the group consisting of a nucleic acid, a protein, a peptide, a sugar, a lipid, an antigen, an antibody, a receptor, a ligands and a cell.
[7]

The operation method according to any one of [1] to [6], wherein the magnetic solid existing in the liquid layer is moved to the liquid layer along the inner wall surface of the hollow tube, and the magnetic particles is dispersed in the liquid layer with movement of the magnetic solid by operating the magnetic field from the outside of the hollow tube.
[8]

The operation method according to [7], wherein the movement of the magnetic solid is a repetitive reciprocating movement in the liquid layer.
[9]

The operation method according to [1] or [8], wherein the magnetic particles having the target substance fixed to the magnetic particles are washed while the target substance is fixed to the magnetic particles by dispersing the magnetic particles on which the target substance is fixed in the first liquid layer in which the first liquid layer is a cleaning liquid layer.
[10]

The operation method according to [7] or [8], wherein the target substance fixed to the magnetic particles is eluted into the eluate by dispersing the magnetic particles on which the target substance is fixed in the second liquid layer in which the second liquid layer is the eluate layer.
[11]

The operation method according to [9], wherein the cleaning liquid is an aqueous solution containing a high concentration of salt or alcohol.
[12]

The operation method according to [10], wherein the eluate is water or a buffer solution containing a low-concentration salt.
[13]

The operation method according to any one of [1] to [12], wherein the gel-like medium is an oil-based gel that is insoluble or sparingly soluble in the liquid constituting the liquid layer.
[14]

The operation method according to any one of [1] to [13], wherein the closed end of the hollow tube comprises a filter that allows the liquid constituting the liquid layer to pass through but prevents the movement of the magnetic particles.
[15]

The operation method according to any one of [1] to [14], wherein magnetic field operation in the third step is performed by movement of the magnetic solid from the second liquid layer to the gel-like medium layer so that the predetermined amount or the total amount of the magnetic particles existing in the second liquid layer is moved into the gel-like medium layer.

### Effect of the Invention

According to the present invention, in a tubular device in which gel-like medium layers and liquid layers are alternately arranged in a hollow tube, magnetic particles and magnetic solids can be removed from the eluate by inverting and moving the mixed magnetic particles and magnetic solids into the gel-like medium layer by magnetic field operation from the outside of the hollow tube from the purified aqueous liquid (eluent) containing the target substance. So, it is not necessary to separately remove the magnetic particles from the eluate by centrifugation or the like. Therefore, the operation of collecting the target substance becomes simple. Also, when the target substance is nucleic acid (DNA or RNA), an eluate that can be immediately subjected to PCR or RT-PCR can be collected.

### SIMPLE EXPLANATION OF DRAWINGS

FIG. 1 is a diagram schematically showing an outline of a method for operating magnetic particles.

FIG. 2 is a diagram schematically showing each step of an embodiment of separating and purifying nucleic acid (DNA).

### EMBODIMENT OF THE INVENTION

### [Device]

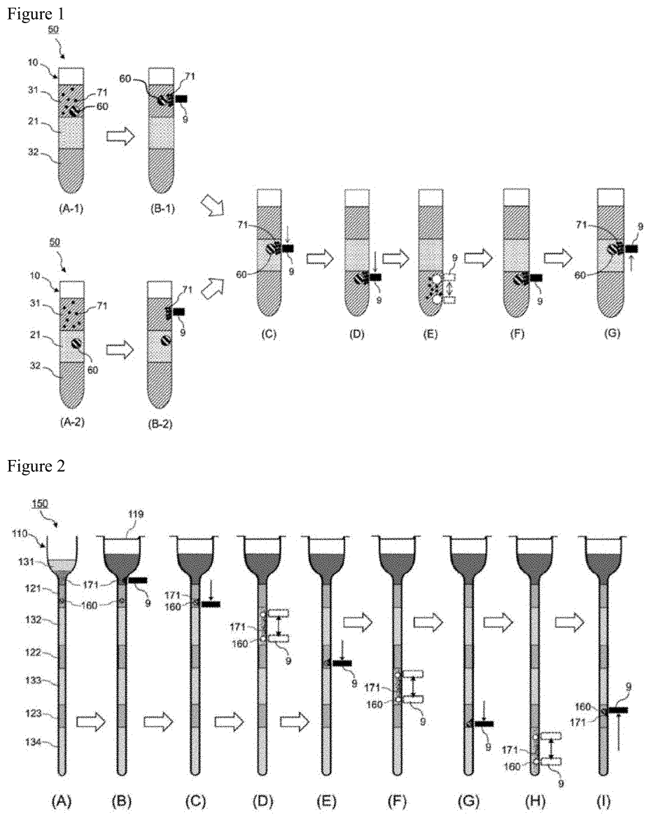

The structure of the device used for operating the magnetic particles in one embodiment of the present invention will be described with reference to FIG. 1 (in the following description, the vertical direction is based on FIG. 1). The hollow tube 10 constituting the device 50 for operating magnetic particles used in the present invention preferably has an upper end open for sample injection, and the open end can be closed from the viewpoint of contamination. The lower end of the hollow pipe 10 is a closed end. Normally, the hollow tube constituting the operation tube has a substantially circular cross section but does not exclude a tube having a cross section of another shape. As shown in FIG. 1 at (A-1), gel-like medium layer and liquid layer are alternately arranged in the hollow tube 10, and second liquid layer 32, gel-like medium layer 21, and first liquid layer 31 is layered and filled in this order from the bottom (closed end) side of the hollow tube 10. A magnetic solid 60 is arranged in the first liquid layer 31 (FIG. 1 at (A-1)). In this case, the magnetic solid 60 may settle in the liquid due to gravity and be placed in contact with the gel-like medium layer 21. Further, the magnetic solid 60 may be arranged in the gel-like medium layer 21 (FIG. 1 at (A-2)). In this case, the magnetic solid 60 is held in the gel-like medium layer 21. Further, in the hollow tube, three or more liquid layers may be devices separated by a gel-like medium layer.

The gel-like medium constituting the gel-like medium layer 21 is preferably immiscible with the liquid layers 31 and 32 in contact with the gel-like medium layer 21 and is insoluble or sparingly soluble in the liquid forming these liquid layers. For example, when the liquid layers 31 and 32 are aqueous liquids, the gel-like medium layer 21 is preferably an oil-based gel that is insoluble or sparingly soluble in the aqueous liquid. Further, the gel-like medium layer is preferably a chemically inert substance. Here, being insoluble or sparingly soluble in a liquid means that the solubility in a liquid at 25° C. is approximately 100 ppm or less. A chemically inert substance is one even when in contact with a liquid layer and magnetic particles, a substance that does not chemically change or affect a liquid layer, magnetic particles, or a substance fixed or adsorbed on the magnetic particles.

Magnetic particles 71 are contained in the first liquid layer 31. The magnetic particle 71 can selectively fix a specific target substance such as nucleic acid on the surface of the particle. Fixation of the target substance to the magnetic particles 71 is performed, for example, in the first liquid layer 31. In this case, the liquid constituting the first liquid layer 31 contains a target substance to be fixed on the surface of the magnetic particles 71. Further, the magnetic particles 71 in which the target substance is fixed on the particle surface in advance may be added to the first liquid layer 31. Also, from the open end of the hollow tube 10 filled with the second liquid layer 32 and the gel-like medium layer 21, the liquid containing magnetic particles 71 in which the target substance is fixed on the particle surface may be injected onto the gel-like medium layer 21.

### [Operation of Magnetic Particles]

As shown in FIG. 1 at (B-1) or (B-2), the magnetic particles 71 are attracted to the inner wall surface of the hollow tube 10 by an external magnetic field operation by the magnet 9. For the magnetic field operation, a magnetic force source such as a permanent magnet (for example, a ferrite magnet and a neodymium magnet) or an electromagnet can be used. The magnetic particles 71 attracted to the inner wall surface of the hollow tube 10 form aggregates, and the liquid constituting the first liquid layer 31 may be taken into the aggregates of the magnetic particles. Further, when the first liquid layer 31 contains impurities other than the target substance, the impurities may also be incorporated into the aggregates of the magnetic particles. In particular, denatured proteins and the like have an action of adhering magnetic particles to each other, and these impurities are also incorporated into aggregates of magnetic particles. On the other hand, the magnetic solid 60 arranged in the first liquid layer 31 is also attracted to the inner wall surface by the magnet 9 and integrated with the agglomerates of the magnetic particles 71 (FIG. 1 at (B-1)). The magnetic solid 60 is a magnetic material having a larger particle size than the magnetic material particles 71.

In the first liquid layer 31, the magnetic solid 60 integrated with the aggregate of the magnetic particles 71 can reciprocate the magnetic particles together with the magnetic solid up and down or up, down, left, and right along the inner wall surface of the hollow tube by the magnetic field operation by the magnet 9, but the movement in the vertical direction (long axis direction of hollow pipe) is preferable. The reciprocating movement of the magnetic particles and the magnetic solid by magnetic field operation can be performed a plurality of times. By this magnetic field operation, the magnetic particles 71 or their aggregates existing around the magnetic solid and between the magnetic solid and the inner wall surface of the hollow tube are dispersed in the first liquid layer 31. Therefore, when the liquid constituting the first liquid layer 31 is a cleaning liquid, impurities such as denatured proteins adsorbed non-specifically on the magnetic particles can be washed and removed. Even when the magnetic solid 60 does not exist in the first liquid layer 31, the magnetic field operation by the magnet 9 can be similarly performed on the magnetic particles.

The first step of the method of the present invention is a step of moving magnetic particles and magnetic solids in the first liquid layer to an adjacent gel-like medium layer by operating a magnetic field from the outside of the hollow tube. In this step, when the magnet 9 outside the hollow tube 10 is moved from the side surface of the first liquid layer 31 to the side surface of the gel-like medium layer 21, when the magnetic solid 60 is arranged in the first liquid layer 31, the magnetic solid 60 integrated with the magnetic particles 71 or an aggregate thereof enters the gel-like medium layer 21 from the first liquid layer 31. Also, even when the magnetic solid 60 is not arranged in the first liquid layer 31, due to the movement of the magnet 9, the magnetic particles 71 or their aggregates similarly enter the gel-like medium layer 21 from the first liquid layer 31. At this time, most of the aqueous liquid physically attached as droplets around the magnetic particles 71 is generated, when the magnetic particles 71 enter the inside from the surface of the gel-like medium layer 21, it is separated from the particle surface and remains in the liquid component of the liquid layer 31. On the other hand, the magnetic particles 71 can easily move in the gel-like medium layer 21 together with the magnetic solid 60 while holding the target substance fixed to the particles.

The gel-like medium is perforated by the entry and movement of the magnetic particles 71 and the magnetic solid 60 into the gel-like medium layer 21, but the gel self-repairs due to its thixotropic property (sway denaturation). When a magnetic particle and a magnetic solid move in the gel by magnetic field operation, when a shearing force is applied, the gel is locally fluidized (viscous) due to its thixotropic property. Therefore, the magnetic particles and the magnetic solid can easily move in the gel while perforating the fluidized portion. After the passage of magnetic particles and magnetic solids, the gel released from the shear force quickly restores its original elastic state. Therefore, through holes are not formed in the portion through which the magnetic particles and the magnetic solid have passed, and the liquid hardly flows into the gel through the perforated portion of the magnetic particles and the magnetic solid.

The restoring force due to the thixotropic properties of the gel as described above acts to squeeze the liquid attached to the magnetic particles 71. Therefore, even when the magnetic particles 71 become aggregates and move into the gel-like medium layer 21 with the droplets incorporated therein, the magnetic particles and the droplets can be separated by the restoring force of the gel. On the other hand, impurities such as modified proteins incorporated into the aggregates of the magnetic particles strongly adhere to each other, so that it is difficult to separate them from the magnetic particles by the restoring force of the gel. Therefore, these impurities move in the gel-like medium layer together with the magnetic solid while being taken into the aggregate of the magnetic particles.

In the case of magnetic solid 60 is arranged in the gel-like medium layer 21, when the magnet 9 outside the hollow tube 10 is moved from the side surface of the first liquid layer 31 to the side surface of the gel-like medium layer 21, the magnetic particles 71 enter the gel-like medium layer 21 from the first liquid layer 31, and the magnetic solid 60 arranged in the gel-like medium layer 21 is attracted to the magnet 9 (FIG. at 1C). Therefore, the magnetic solid 60 moves in the gel-like medium layer together with the magnetic particles 71 or their aggregates.

The second step of the method of the present invention is a step of moving the magnetic particles and the magnetic solid moved into the gel-like medium layer into the second liquid layer by operating a magnetic field from the outside of the hollow tube. In this step, the magnetic particles 71 and the magnetic solid 60 that have passed through the gel-like medium layer 21 move from the gel-like medium layer 21 to the second liquid layer 32 by the magnetic field operation (FIG. 1 at (D)). As mentioned above, in the gel-like medium, through holes are not formed in the portion through which the magnetic particles and the magnetic solid have passed, so that the inflow of liquid from the first liquid layer 31 to the second liquid layer 32 hardly occurs. On the contrary, the inflow of the liquid from the second liquid layer 32 into the first liquid layer 31 hardly occurs.

When the magnet 9 is moved along the side surface of the second liquid layer 32, the magnetic solid 60 and the magnetic particle 71 also move in the second liquid layer as the magnet 9 moves. The magnet 9 can be reciprocated up and down or up, down, left, and right along the outer wall surface of the hollow tube but is preferably moved in the vertical direction (longitudinal direction of the hollow tube). The reciprocating movement of the magnet 9 can be performed a plurality of times. When the magnetic solid 60 and the magnetic particles 71 move or move due to the magnetic field operation by the movement of the magnet 9, the magnetic particles 71 forming the agglomerates are dispersed in the second liquid layer (FIG. 1 at (E)).

Therefore, when the liquid constituting the second liquid layer 32 is a cleaning liquid, impurities such as denatured proteins adsorbed non-specifically on the magnetic particles can be washed and removed. Also, in the case of liquid constituting the second liquid layer 32 is an eluate, when the magnetic particles are dispersed, since the target substance such as nucleic acid immobilized on the magnetic particles is eluted (freed) in a state where the magnetic particles are sufficiently exposed to the eluate, the recovery efficiency of the target substance is improved.

The principle that the magnetic particles are dispersed by moving the magnetic particles together with the magnetic solid in the liquid layer is not always clear. However, in the case of visually observing the movement of magnetic solids and magnetic particles, when the magnetic solid 60 moves along the wall surface of the hollow tube 10, it is recognized that the magnetic solid vibrates slightly due to the frictional resistance between the inner wall surface of the hollow tube and the magnetic solid and the delay in the follow-up movement of the magnetic solid with respect to the movement of the magnet. It is presumed that magnetic particles are rapidly dispersed in the liquid layer because micro-vibration of this magnetic solid has the effect of dispersing magnetic particles or the aggregate present around magnetic solid, or micro-vibration of magnetic solid has the effect of disrupting aggregates of magnetic particles present between the inner wall surface of the hollow tube and the magnetic solid.

Third step of the method of the present invention is a step of reversing and moving the magnetic particles and the magnetic solid moved into the second liquid layer into the gel-like medium layer by operating a magnetic field from the outside of the hollow tube. In this step, magnetic particle 71 and magnetic solid 60 dispersed in the second liquid layer 32 is attracted to and aggregates on the inner wall surface of the hollow tube 10 by slowly moving the magnet 9 up and down or up, down, left, and right, and then gradually stopping it (FIG. 1 at (F)). Operating speed of magnet 9 is the speed at which magnetic particle 71 and magnetic solid 60 is allowed to assemble, which can be adjusted by those skilled in the art by visually observing the movement of the magnetic solid and magnetic particles. Since the magnetic solid and the magnetic particles are black to brown, they can be visually recognized.

By the operation of the magnet 9 outside the hollow tube, by inverting and moving the magnet 9 from the side surface of the second liquid layer 32 to the side surface of the gel-like medium layer 21, a predetermined amount or the whole amount of magnetic particle 71 and magnetic solid 60 gathered on the side of the second liquid layer re-enters the gel-like medium layer from the second liquid layer with movement of magnet 9, and reversely moves to the gel-like medium layer (FIG. 1 at (G)). Here, the magnetic field operation of the magnet 9 from the outside of the hollow tube is performed at an operating speed so that the aggregated magnetic particles 71 and the magnetic solid 60 are not dispersed again in the second liquid layer. The speed can be adjusted by visually observing the movement of the magnetic solid and the magnetic particles. Since the magnetic solid and the magnetic particles are black to brown, they can be visually recognized. In addition, it should be noted, the predetermined amounts of the magnetic particles 71 and the magnetic solid 60 are as described below, the amount of the remaining magnetic particles 71 and the magnetic solid 60 (particularly, magnetic particles having a smaller particle size) does not need to be removed by centrifugation or the like.

In this way, the magnetic particles 71 and the magnetic solid 60 can be removed from the second liquid layer 32 by operating the magnetic field from the outside. Therefore, when the liquid constituting the second liquid layer 32 is an eluate that elutes (frees) the target substance fixed on the surface of the magnetic particles, it is not necessary to separately remove the magnetic particles and the magnetic solid from the eluate by centrifugation or the like. Therefore, the operation of collecting the target substance becomes simple. Further, even when the eluate containing the target substance is directly subjected to the next detection process or measurement reaction, the magnetic particles and/or magnetic solids do not interfere with the detection process or measurement reaction. For example, when the target substance is nucleic acid (DNA or RNA), and subjected to the immediate PCR or RT-PCR, no interference with the reaction and measurement occurs.

### [Magnetic Particles]

The magnetic particles 71 used in the present invention are the material which can perform operations such as aggregation, dispersion, and movement in a liquid or a gel-like medium by the action of a magnetic field. Examples of the magnetic material include ferromagnetic metals such as iron, cobalt and nickel, and compounds, oxides and alloys thereof. Specific examples thereof include magnetite (Fe3O4), hematite (Fe2O3, or αFe2O3), maghemite (γFe2O3), titanomagneties (xFe2TiO4.(1−x) Fe3O4, ilmenohematite (xFeTIO3.(1−x) Fe2O3, pyrotite (Fe1-xS (x=0-0.13) Fe7S8 (x-0.13)), greigite (Fe3S4), goethite (αFeOOH), chromium oxide (CrO2), permalloy, alnico magnet, stainless steel, samarium magnet, neodymium magnet, barium magnet.

To facilitate the particle operation in the liquid and the gel-like medium, the particle size of the magnetic particles is preferably about 0.1 to 20 μm, more preferably about 0.5 to 10 μm. The shape of the magnetic particles is preferably spherical with a uniform particle size, but as long as the particles can be operated by magnetic force, the magnetic particles may have an irregular shape and may have a certain particle size distribution. The constituent components of the magnetic particles may be a single substance or may be composed of a plurality of components.

The magnetic particles are preferably those capable of selectively fixing a specific target substance. As long as the target substance can be retained on the surface of the particles or inside the particles, the immobilization method is not particularly limited, and various known immobilization mechanisms such as physical adsorption and chemical adsorption can be applied. For example, various intermolecular forces such as van der Waals force, hydrogen bond, hydrophobic interaction, ion-ion interaction, and π-π stacking fix the target substance on the surface or inside of the particle.

Examples of the target substance immobilized on the magnetic particles include biological substances such as nucleic acids, proteins, peptides, sugars, lipids, antigens, antibodies, receptors and ligands, and cells. When the target substance is a biological substance, the target substance may be fixed on the particle surface by molecular recognition or the like. For example, when the target substance is nucleic acid, the nucleic acid can be selectively adsorbed on the particle surface by using silica-coated magnetic particles. Also, when the target substance is an antibody (for example, a labeled antibody), a receptor, an antigen, a ligand, or the like, the target substance can be selectively fixed to the particle surface by amino group, carboxyl group, epoxy group, avidin, biotin, digoxigenin, protein A, protein G and the like on the particle surface.

As magnetic particles, the substance for selectively fixing the target substance, for example, a compound having various functional groups, silica, streptavidin, staphylococcus aureus, protein A, protein G, immunoglobulin or the like attached to the particle surface of the above magnetic material or a substance coated with these substances is preferably used. As such magnetic particles, commercially available products such as Dynabeads (registered trademark) sold by Life Technologies and MagExtractor (registered trademark) sold by Toyobo can also be used.

The magnetic particles contained in one device are appropriately determined according to the amount of the sample added to the device, the volume of each liquid layer, and the like. For example, when an elongated cylindrical capillary hollow tube having an inner diameter of about 1 to 2 mm is used, amounts of magnetic particles added to the device is usually preferably in the range of about 10 to 200 μg.

### [Magnetic Solid]

The material of the magnetic solid 60 used in the present invention is not particularly limited as long as it is a magnetic material, and similar to those exemplified as the magnetic material constituting the magnetic material particles 71, ferromagnetic metals such as Iron, Cobalt, Nickel and their compounds, oxides, alloys and the like can be mentioned. The shape of the magnetic solid is not particularly limited, and may be spherical, polyhedral, flat, rod-shaped, or the like.

The magnetic solid 60 preferably has a larger particle size than the magnetic particles. In the case of the magnetic solid is non-spherical, the major axis is regarded as the particle size. The particle size of the magnetic solid is preferably 100 μm or more, more preferably 300 μm or more, and even more preferably 500 μm or more. Even when the magnetic particles 71 form aggregates, if the magnetic solid 60 having a particle size larger than that of the magnetic particles coexists, the magnetic particles can be dispersed in the liquid by moving the magnetic solid integrated with the aggregates of the magnetic particles by magnetic field operation. The particle size of the magnetic solid is preferably 10 times or more, more preferably 20 times or more, still more preferably 30 times or more, and particularly preferably 50 times or more the particle size of the magnetic particles.

The upper limit of the particle size of the magnetic solid is not particularly limited as long as it can move in the hollow tube. For example, when the hollow tube is tubular and the magnetic solid is spherical, the particle size of the magnetic solid may be smaller than the inner diameter of the hollow tube. From the viewpoint of facilitating operation by a magnetic field, the particle size of the magnetic solid is preferably 10 mm or less, more preferably 5 mm or less, further preferably 3 mm or less, and particularly preferably 1.5 mm or less. The particle size of the magnetic solid is preferably 100,000 times or less, more preferably 50,000 times or less, still more preferably 10,000 times or less, the particle size of the magnetic particles. In the embodiment shown in FIG. 1, one magnetic solid 60 is used in the hollow tube 10, but a plurality of magnetic solids can also be used.

As the magnetic solid, commercially available metal balls such as iron balls for ball bearings and stainless steel-spheres can be used as they are. It is also possible to impart functionality to the magnetic solid. For example, by coating the surface of a metal material such as iron and stainless steel, corrosion resistance to reagents and samples can be imparted.

In particular, when the magnetic solid is in contact with the aqueous liquid or gel-like medium constituting the liquid layer for a long time in the device for operating magnetic particles, metals such as iron constituting the magnetic solid may corrode, and the corrosive component (for example, metal ions eluted in the liquid layer) may affect the fixation of the target substance and the subsequent reaction with the reagent and sample (for example, enzyme reaction and antigen-antibody reaction), elution of the target substance, and the like. On the other hand, when the magnetic solid has a protective layer (coating layer) on the metal surface for preventing corrosion, the influence of corrosion of the metal can be suppressed.

When forming a protective layer to make the metal surface corrosion resistant, protective layer material is not particularly limited as long as it prevents corrosion of the metal by the aqueous liquid constituting gel medium and liquid layer and may be an inorganic material such as a metal or a metal oxide, or a resin material. Examples of the metal material include gold, titanium, platinum and the like. Examples of the resin material include a fluorine-based resin such as tetrafluoroethylene and an epoxy-based resin. Further, as the protective layer material, a material having little influence on the inhibition of the reaction with the reagent and the sample and the fixation and elution of the target substance is preferably used.

The method of forming the protective layer on the metal surface is not particularly limited. For example, when a metal coating such as gold, titanium, or platinum is applied to give corrosion resistance to a metal surface, a plating method, or a dry process (vapor deposition, sputter, CVD, etc.) is preferably adopted. When a resin coating is applied to the metal surface, a wet coating is preferably adopted.

If the protective layer for preventing corrosion of the metal is peeled off or cracked due to a physical impact or the like, the metal may be exposed and the metal may be corroded from the exposed portion. Therefore, the thickness of the protective layer is preferably about several nm to several hundred μm. In order to make the protective layer so thick, it is preferable to form a resin layer by wet coating. As the resin material, a resin solution, a liquid adhesive, or the like can be used. As the liquid adhesive, a commercially available adhesive for metals may be used as it is. For example, since the two-component curable epoxy adhesive can be cured at room temperature and a protective layer having the above thickness can be easily formed, it is suitably used as a protective layer material for preventing metal corrosion.

When the resin solution is dried and cured by wet coating, it is preferable to set drying conditions so that the protective layer does not peel off. For example, if the magnetic solid after coating is allowed to dry or harden, it is preferable to allow the magnetic solid after coating to stand on a material to which the resin material does not easily adhere or a material having solvent resistance to the solvent of the coating liquid.

A coating layer other than the corrosion-resistant coating may be provided on the surface of the magnetic solid. For example, the surface of the magnetic solid may be coated with various functional molecules so that a substance different from the substance fixed to the magnetic particles is fixed to the surface of the magnetic solid. In addition, the surface of the magnetic solid may be coated with an optical material such as a light emitting substance and a fluorescent substance. According to such a configuration, since the position of the magnetic solid can be detected optically, for example, when automating the operation of magnetic particles, it can be applied to the position detection and position correction of the magnetic solid and the magnetic particles integrated with the magnetic solid. Further, by adjusting the material, size, and shape of the magnetic solid, the magnetic solid can also function as an actuator for valve and pump operation by magnetic field operation in the microchannel system. In addition, a magnetic solid can be used as a heat source for a chemical reaction as a receptor for driving power of a fluid control element by magnetic resonance or as a heating element by electromagnetic induction.

In the embodiment shown in FIG. 1, FIG. 1 at (A-1) illustrates an embodiment in which the magnetic solid 60 is previously arranged in the first liquid layer 31, further, FIG. 1 at (A-2) illustrates an embodiment in which the magnetic solid 60 is arranged in the gel-like medium layer 21 in advance. In addition to these examples, the magnetic solid 60 may be pre-located in the second liquid layer 32. Further, the magnetic solid may be charged into the first liquid layer 31, or the magnetic solid may be placed on the gel-like medium layer 21 and the liquid may be injected therein. Further, the magnetic solid may be charged into the hollow tube together with the magnetic particles and the liquid. In the embodiment shown in FIG. 1, one magnetic solid is used for one device, but a plurality of magnetic solids may be used.

### [Hollow Tube]

In the present invention, the magnetic particles and the magnetic solid are operated by the liquid layer and the gel-like medium layer filled in the hollow tube 10. The material and shape of the hollow tube are not particularly limited as long as they can move magnetic particles and magnetic solids in the hollow tube 10 by magnetic field operation from outside the tube and can hold a liquid and a gel-like medium. For example, a structure or the like in which another flat plate material is bonded to the upper surface of the flat plate material having a linear groove having a straight tubular structure (capillary) with an inner diameter of about 0.1 mm to 5 mm, preferably an inner diameter of about 1 to 2 mm and a length of about 50 to 200 mm, or a width of about 1 to 2 mm and a depth of about 0.5 to 1 mm and a length of about 50 mm to 200 mm can be used.

If the size of the hollow tube is made as small as possible, it can be used as a microdevice for operating a micro liquid or a chip for operating a micro liquid. The shape of the hollow tube is not limited to a tubular shape and a planar shape, and the movement path of the particles may be a structure having branches such as a cross or a T shape.

Since the device used in the present invention can move the magnetic particles 71 and the magnetic solid 60 in the hollow tube 10 by the magnetic field operation, the device after adding the sample can be a closed system. If the device is a closed system, contamination from the outside can be prevented. Therefore, it is particularly useful when an easily degradable substance such as RNA is fixed to magnetic particles and operated. If the device is a closed system, the opening of the hollow tube can be sealed using a method of heat fusion or other suitable sealing means. When it is necessary to take out the particles and the aqueous liquid after the operation to the outside of the device, the opening can be removably sealed by using a resin stopper or the like. In addition, the closed end of the lower end of the hollow pipe is temporarily used as the open end, and a filter (For example, Membrane filter) having a pore size capable of blocking the passage of the magnetic particles 71 and the magnetic solid 60 may be adhered to the end portion, and then a removable sealing plug may be attached to form a closed end. Such a filter blocks the movement of the magnetic particles 71 and the magnetic solid 60 but allows the aqueous liquid (eluent) constituting the liquid layer in contact with the filter to pass through. Therefore, by removing the sealing plug, it is possible to recover the eluate containing no magnetic particles.

The material of the hollow tube 10 is not particularly limited as long as it does not shield the magnetic field from the outside. Examples thereof include polyolefins such as polypropylene and polyethylene, fluororesins such as tetrafluoroethylene, and water-repellent resin materials such as polyvinyl chloride, polystyrene, polycarbonate and cyclic polyolefins. Since the inner wall surface of the hollow tube serves as a transport surface for magnetic particles and magnetic solids, if the inner wall surface is water repellent, the separation of the liquid from the magnetic particles is promoted, and the solid-liquid separation can be made highly efficient. In addition to these materials, ceramics, glass, silicones, metals and the like may be used. In order to enhance the water repellency of the inner wall surface of the hollow tube, a coating treatment with a fluororesin, silicone or the like may be performed.

Since the inner wall surface of the hollow tube serves as a transport surface for magnetic particles, it is preferably a smooth surface, and in particular, the surface roughness is preferably Ra=0.1 μm or less. For example, the magnet 9 is brought close to the tube from the outside of the tube, and the magnetic particles move while being pressed against the transport surface by magnetic field fluctuation, but by having surface roughness of the conveyed surface is Ra=0.1 μm or less, it is possible to sufficiently provide magnetic particles' followability to a fluctuating magnetic field.

When optical measurement such as changes in absorbance, fluorescence, chemiluminescence, bioluminescence, and refractive index is performed or light irradiation is performed during or after the operation of the magnetic particles and the magnetic solid, a hollow tube having light transmission is preferably used. Further, if the hollow tube is light transmissive, it is preferable because the situation where the particles in the hollow tube are operated by a magnetic field can be visually recognized. On the other hand, when it is necessary to shield liquid and magnetic particles from light, a hollow tube made of metal or the like that does not transmit light is preferably used. Depending on the purpose of use and the like, a hollow tube having a light transmitting portion and a light shielding portion can also be adopted.

### [Liquid Layer]

The liquid constituting the first liquid layer 31 contains a target substance to be separated and purified. Examples of the target substance include biological substances such as nucleic acids, proteins, peptides, sugars, lipids, antigens, antibodies, receptors, ligands and cells. The liquid constituting the liquid layer 31 contains impurities in addition to the target substance. For example, when separating and purifying nucleic acid from blood, the liquid layer 31 contains a wide variety of impurities such as proteins and sugars eluted from cells in addition to the nucleic acid which is the target substance.

The liquid constituting the first liquid layer 31 is, in one embodiment, a mixture of a biological sample such as blood and a solution for extracting a target substance from the sample. The type of liquid is not particularly limited, but one that does not dissolve the gel-like medium is preferable. As the liquid, an aqueous solution and an aqueous liquid such as a mixed solution composed of water and an organic solvent are generally preferably used. Examples of the solution for extracting the target substance include a cell lysate. The cell lysate contains components capable of lysing cells such as chaotropic substances and surfactants. Examples of the chaotropic salt include guanidine hydrochloride, guanidine isocyanate, potassium iodide, urea and the like. Chaotropic salts are potent protein denaturants, which have the function of lysing the protein of cells and releasing the nucleic acid in the cell nucleus into the liquid, and also have the effect of suppressing the action of nucleic acid degrading enzymes. In addition to the above, the liquid constituting the first liquid layer 31 may contain various buffers, salts, and other various auxiliary agents, an organic solvent such as alcohol, and the like.

In one embodiment, the magnetic particles 71 selectively fix a specific target substance on the particle surface in the first liquid layer 31. When the magnetic particles 71 are dispersed in the first liquid layer 31, the magnetic particles 71 are washed while maintaining the state in which the target substance is fixed. Therefore, the liquid constituting the first liquid layer 31 also functions as a cleaning liquid. For example, if the target substance is nucleic acid, the cleaning solution may be any as long as it can release components other than nucleic acids (for example, proteins, sugars, etc.) adhering to the magnetic particles, reagents used for treatments such as nucleic acid extraction, and the like in the cleaning solution, while the nucleic acid remains fixed to the surface of the magnetic particles. Examples of the cleaning liquid include a high salt concentration aqueous solution such as sodium chloride, potassium chloride and ammonium sulfate, and an alcohol aqueous solution such as ethanol and isopropanol.

A device in which a plurality of layers of the first liquid layer 31 are separated by a gel-like medium layer 21 can also be used. When the first liquid layer 31 is filled with two layers, in the first “first liquid layer 31” adjacent to the opening of the hollow tube 10 (upper end of the hollow tube), the target substance is extracted and the target substance is fixed to the magnetic particles 71. Since the first liquid layer 31 is also a cleaning liquid layer, the magnetic particles 71 are cleaned at the same time. After that, by operating the magnetic field, the magnetic particles 71 and the magnetic solid 60 move from the first “first liquid layer 31” to the second “first liquid layer 31” via the adjacent gel-like medium layer. When the magnetic particles 71 are redispersed in this liquid layer, the magnetic particles 71 are washed while the target substance is kept fixed, so that residual impurities are removed and the target substance is purified. The cleaning operation described above can be repeated by further adding the first liquid layer 31. When a plurality of “first liquid layer 31” are filled in the hollow tube 10, the liquids constituting each liquid layer may be the same or different from each other.

The layer length of the first liquid layer 31 can be appropriately determined by those skilled in the art in consideration of the inner diameter and length of the hollow tube, the amount of the sample to be injected into the device, and the like. For example, by operating the magnetic field with the magnet 9, it is sufficient to determine the layer length at which the magnetic solid 60 integrated with the aggregate of the magnetic particles 71 is appropriately moved in the liquid layer, and as a result, the magnetic particles can be well dispersed. Such a layer length is 0.5 to 30 mm in one embodiment and 3 to 10 mm in the other embodiment.

By operating the magnetic field of the magnet 9 from the outside of the hollow tube, the magnetic particles 71 move from the adjacent gel-like medium layer 21 to the second liquid layer 32 with the target substance fixed on the surface thereof. The liquid constituting the second liquid layer 32 is an eluate that elutes (frees) or separates the target substance fixed to the particles from the magnetic particles 71, and preferably does not dissolve the gel-like medium. When the target substance immobilized on the magnetic particles is nucleic acid, the eluate is preferably water (distilled water, purified water, etc.) or a buffer solution containing a low concentration salt (for example, sodium chloride). As the buffer solution, a phosphate buffer solution, a Tris buffer solution, or the like can be used, but generally, a 5 to 20 mM Tris buffer solution adjusted to pH 7 to 9 is used.

When the second liquid layer 32 is an eluent layer of the target substance, the layer length or volume can be appropriately determined by those skilled in the art in consideration of the inner diameter and length of the hollow tube, the amount of sample to be injected into the device, and the like. For example, by manipulating the magnetic field with the magnet 9, the layer length or volume at which the magnetic solid 60 integrated with the agglomerates of the magnetic particles 71 is appropriately moved in the liquid layer, and as a result, the magnetic particles can be well dispersed, may be determined. Such a volume is 1 μL to 1000 μL in one embodiment and 50 μL to 300 μL in another embodiment. If the volume of the eluate is constant, the recovery rate, concentration, etc. of biological components such as nucleic acids recovered in the eluate can be accurately calculated.

### [Gel-Like Medium Layer]

The gel-like medium forming the gel-like medium layer 21 may be in the form of a gel or a paste before the operation of the magnetic particles 71 and the magnetic solid 60 by the magnet 9. The gel-like medium is preferably an oil-based gel that is insoluble or sparingly soluble in the liquids constituting the first liquid layer 31 and the second liquid layer 32 and is a substance that is chemically inert.

The material and composition of the gel-like medium constituting the gel-like medium layer are not particularly limited. The gel-like medium is formed by adding a gelling agent to a water-insoluble or poorly water-soluble liquid substance such as liquid oil, ester oil, hydrocarbon oil, or silicone oil to gel. The gel (physical gel) formed by the gelling agent forms a three-dimensional network due to a weak intermolecular binding force such as hydrogen bond, van der Waals force, hydrophobic interaction, electrostatic attraction, and reversibly undergoes a sol-gel transition by an external stimulus such as heat. As the gelling agent, hydroxy fatty acid, dextrin fatty acid ester, glycerin fatty acid ester and the like are used. Amount of gelling agent used can be appropriately determined with respect to 100 parts by weight of the water-insoluble or poorly water-soluble liquid substance, for example, in the range of 0.1 to 5 parts by weight in consideration of the physical characteristics of the gel and the like.

The method of gelation is not particularly limited. For example, a physical gel is formed by heating water-insoluble or poorly water-soluble liquid substance, adding a gelling agent to the heated liquid substance, completely dissolving the gelling agent, and then cooling to a sol-gel transition temperature or lower. The heating temperature can be appropriately determined in consideration of the physical properties of the liquid substance and the gelling agent.

Further, a hydrogel material (for example, gelatin, collagen, starch, pectin, hyaluronic acid, chitin, chitosan, alginic acid, or derivatives thereof, etc.) prepared by equilibrium swelling in a liquid can also be used as a gel-like medium. As the hydrogel, those obtained by chemically cross-linking a hydrogel material, those obtained by gelling with a gelling agent (for example, salts of alkali metals and alkaline earth metals such as lithium, potassium and magnesium, salts of transition metals such as titanium, gold, silver and platinum, and silica, carbon, and alumina compounds, etc.) or the like can also be used.

### [Preparation of Gel-Like Medium Layer and Liquid Layer in Hollow Tube]

To prepare the gel-like medium layer and the liquid layer in the hollow tube, the filling of the gel-like medium and the liquid in the hollow tube can be performed by any method. Prior to filling the hollow tube, it is preferable that the opening at one end of the hollow tube is sealed to form a closed end, and the liquid and gel-like medium are sequentially filled from the opening at the other end. When loading a gel-like medium into a capillary hollow tube with an inner diameter of about 1 to 2 mm, for example, filling is performed by attaching a metal injection needle to an Arlock type syringe and discharging a gel-like medium to a predetermined position in the capillary.

The volumes of the liquid and gel-like medium filled in the hollow tube can be appropriately set according to amounts of magnetic particles to be operated, the type of operation, and the like. When a plurality of gel-like medium layers and liquid layers are provided in the hollow tube, the layer length or volume of each layer may be the same or different.

The filling of the liquid and the gel-like medium into the hollow tube may be performed immediately before the operation of the magnetic particles and the magnetic solid by the magnet 9 or may be performed after a long time before the operation. When the gel-like medium is insoluble or sparingly soluble in the liquid, there is little interaction and absorption between the gel-like medium and the liquid even after a long period of time after filling. The device used in the present invention may be in a state in which a liquid and a gel-like medium are pre-filled in a hollow tube. Further, the magnetic particles and the magnetic solid may be arranged in advance in the device.

When a plurality of gel-like medium layers are produced in one hollow tube, the gel-like media to be filled may have the same composition or different compositions. For example, when treating or reacting by heating in some of the liquid layers among a plurality of liquid layers, only for the gel-like medium layer adjacent to the liquid layer, the gel-like medium having a high sol-gel transition point capable of maintaining a gel state or a gel-sol intermediate state even at the temperature required for the heating was used and, for the other gel-like medium layer, a gel-like medium having a relatively low sol-gel transition point can be used. In addition, a gel-like medium having appropriate characteristics can be appropriately selected according to the characteristics and volume of the liquid constituting the adjacent liquid layer.

### EXAMPLE

Next, the present invention will be described in more detail with reference to examples, but the scope of the present invention is not limited thereto.

### Example 1

### [Manufacturing a Device]

Hollow tube 110 (Inner diameter 1.8 mm) made of polypropylene was filled with second liquid layer (nucleic acid eluate) 134 (1 mM EDTA, 10 mM Tris-hydrochloric acid buffer, pH 8.0) 200 μL; gel-like medium layer 123; third “first liquid layer” (cleaning liquid) 133 (0.5M NaCl, 70% ethanol) 200 μL, gel-like medium layer 122; second “first liquid layer” (cleaning liquid) 132 (4 mM guanidine hydrochloride, 30% ethanol) 200 μL; gel-like medium layer 121; and the first “first liquid layer” (nucleic acid extract) 131 (4 mM guanidine hydrochloride, 5% (w/v) Triton X-100, 50 mM Tris-hydrochloric acid buffer, pH 7.0) in order from the lower end (See FIG. 2 at (A)).

The gel-like medium layers 121 to 123 were filled with gel (manufactured by Shin-Etsu Chemical Co., Ltd., trade name “KSG-15”) so that the layer length of each layer was about 1 cm. While filling the hollow tube 110 with the gel-like medium layer 121, a metal sphere 160 having a particle size of 1.2 mm made of SUS403 was placed in the gel-like medium layer 121. Further, magnetic beads 171 (silica-coated magnetic beads (average particle size 3 μm) for nucleic acid extraction attached to Toyobo's nucleic acid extraction kit “MagExtractor™-Genome” are resuspended in distilled water) were added into the first liquid layer (nucleic acid extract) 131. The amount of the magnetic beads 171 used was 500 μg.

In this way, as shown in FIG. 2 at (A). A nucleic acid extraction particle manipulation device 150 was prepared in which Four aqueous liquid layers 131, 132, 133 and 134, and three gel-like medium layers 121, 122 and 123 were alternately layered, a metal sphere 160 which was a magnetic solid was provided in a gel-like medium layer 121, and magnetic beads 171 were provided in a nucleic acid extract 131.

### [DNA Extraction Operation]

50 μL of human whole blood to which EDTA was added as an anticoagulant was added into the first liquid layer (nucleic acid extract) 131 through the opening of the hollow tube 110. DNA was liberated and adsorbed on the surface of the magnetic beads 171 by sufficiently stirring the blood and magnetic particles in the first liquid layer (nucleic acid extract) 131 using a micropipette. Then, the opening of the hollow tube 110 was closed with a lid, and the hollow tube 110 was sealed.

Next, neodymium magnet (cylindrical shape with a diameter of 6 mm and a length of 23 mm, trade name “NE127” manufactured by 26 Seisakusho) was brought close to the side surface of the hollow tube 110, and the magnetic beads 171 in which the DNA was fixed on the surface in the first liquid layer (nucleic acid extract) 131 were assembled on the inner wall surface of the hollow tube 110 near the magnet 9 (FIG. 2 at (B)). Then, the magnet 9 was moved from the side surface of the first liquid layer (nucleic acid extract) 131 toward the side surface of the gel-like medium layer 121, and the magnetic beads 171 were allowed to enter the gel-like medium layer 121. At that time, the metal sphere 160 arranged in the gel-like medium layer 121 was also attracted to the magnet 9 together with the magnetic beads 171 (FIG. 2 at (C)).

The magnet 9 is moved from the side surface of the gel-like medium layer 121 toward the side surface of the first liquid layer (cleaning liquid) 132, the metal sphere 160 was moved into the first liquid layer (cleaning liquid) 132 with the magnetic beads 171. When the magnet 9 is reciprocated in the long axis direction of the hollow tube along the side surface of the first liquid layer (cleaning liquid) 132, it was visually confirmed that the metal sphere 160 moved following the magnet 9 and the magnetic beads 171 were dispersed in the first liquid layer (cleaning liquid) 132 (FIG. 2 at (D)).

Then follow the same procedure, by moving the magnet 9 along the side surface of the hollow tube 110, the metal sphere 160 and the magnetic beads 171 in the first liquid layer (cleaning liquid) 132 are allowed to enter the gel-like medium layer 122 (FIG. 2 at (E)), further, the magnet 9 was reciprocated in the long axis direction of the hollow tube 110 in the first liquid layer (cleaning liquid) 133 to disperse the magnetic beads 171 in the cleaning liquid (FIG. 2 at (F)). After that, by moving the magnet 9 along the outer wall surface of the hollow tube 110, the metal sphere 160 and the magnetic beads 171 were allowed to enter the gel-like medium layer 123 (FIG. 2 at (G)) and further moved into the second liquid layer (nucleic acid eluate) 134. Next, magnetic beads are dispersed in the nucleic acid eluate 134 by reciprocating the magnet 9 along the long axis direction of the hollow tube on the side surface of the second liquid layer (nucleic acid eluate) 134, and DNA was released from the surface of the magnetic beads 171 and eluted into the nucleic acid eluate (FIG. 2 at (H)).

After that, the magnet 9 was slowly moved along the side surface of the hollow tube 110, and it was visually confirmed that all the magnetic beads 171 and the metal sphere 160 had gathered on the inner wall surface of the hollow tube 110. Next, by slowly moving the magnet 9 toward the opening (upper end) of the hollow tube 110, the metal sphere 160 integrated with the magnetic bead 171 from which the DNA was released was inverted and moved into the gel-like medium layer 123 (FIG. 2 at a(I)). At that time, the magnetic beads 171 and the metal sphere 160 were inverted and moved to the vicinity of the upper end of the gel-like medium layer 123 so that they would not fall into the second liquid layer (nucleic acid eluate) 134. It was visually confirmed that the magnetic beads 171 did not remain in the second liquid layer (nucleic acid eluate) 134. Finally, a notch was made in the polypropylene tube surface of the nucleic acid eluate 134 near the interface with the gel-like medium layer 123 using a cutter knife, the tube was broken, and the nucleic acid eluate 134 was recovered from the opening. The nucleic acid eluate collected in the tube was visually observed again, but no mixture of magnetic beads was observed.

### EXPLANATION OF SYMBOLS

50: Device
10: Hollow tube
21: Gel-like medium layer
31: First liquid layer
32: Second liquid layer
60: Magnetic solid
71: Magnetic particles
9: Magnet
150: Particle operation device for nucleic acid extraction
110: Hollow tube
121-123: Gel-like medium layer (silicone gel)
131: First liquid layer (nucleic acid extract)
132, 133: First liquid layer (cleaning liquid)
134: Second liquid layer (nucleic acid eluate)
160: Magnetic solid (metal sphere)
171: Magnetic particles (magnetic beads)

1. An operation method of magnetic particles, in a device wherein a gel-like medium layer and a liquid layer are alternately arranged in a hollow tube having an open end which may be closed on one side and a closed end on the other side, and the first liquid layer and second liquid layer are separated by a gel-like medium layer, and a magnetic particle and a magnetic solid having a particle size larger than that of the magnetic particle are present in the first liquid layer;
which comprises
the first step of moving the magnetic particles and the magnetic solid in the first liquid layer to the gel-like medium layer by operating a magnetic field from the outside of the hollow tube;
the second step of moving the magnetic particles and the magnetic solid which were moved into the gel-like medium layer into the second liquid layer by operating a magnetic field from the outside of the hollow tube; and,
the third step of reversing and moving the magnetic particles and the magnetic solid which were moved into the second liquid layer into the gel-like medium layer by operating a magnetic field from the outside of the hollow tube.

2. The operation method according to claim 1, wherein the magnetic solid has a particle size of 100 μm or more.

3. The operation method according to claim 1, wherein the particle size of the magnetic solid is 10 times or more the particle size of the magnetic particles.

4. The operation method according to claim 1, wherein the magnetic solid has a protective layer on the surface of the magnetic solid to prevent corrosion in the liquid layer.

5. The operation method according to claim 1, wherein the magnetic particles are particles capable of selectively fixing a target substance in a sample which is introduced into the hollow tube to the particle surface.

6. The operation method according to claim 5, wherein the target substance is at least one selected from the group consisting of a nucleic acid, a protein, a peptide, a sugar, a lipid, an antigen, an antibody, a receptor, a ligand and a cell.

7. The operation method according to claim 1, wherein the magnetic solid existing in the liquid layer is moved to the liquid layer along the inner wall surface of the hollow tube, and the magnetic particles is dispersed in the liquid layer with movement of the magnetic solid by operating the magnetic field from the outside of the hollow tube.

8. The operation method according to claim 7, wherein the movement of the magnetic solid is a repetitive reciprocating movement in the liquid layer.

9. The operation method according to claim 7, wherein the magnetic particles having the target substance fixed to the magnetic particles are washed while the target substance is fixed to the magnetic particles by dispersing the magnetic particles on which the target substance is fixed in the first liquid layer in which the first liquid layer is a cleaning liquid layer.

10. The operation method according to claim 7, wherein the target substance fixed to the magnetic particles is eluted into the eluate by dispersing the magnetic particles on which the target substance is fixed in the second liquid layer in which the second liquid layer is the eluate layer.

11. The operation method according to claim 9, wherein the cleaning liquid is an aqueous solution containing a high concentration of salt or alcohol.

12. The operation method according to claim 10, wherein the eluate is water or a buffer solution containing a low-concentration salt.

13. The operation method according to claim 1, wherein the gel-like medium is an oil-based gel that is insoluble or sparingly soluble in the liquid constituting the liquid layer.

14. The operation method according to claim 1, wherein the closed end of the hollow tube comprises a filter that allows the liquid constituting the liquid layer to pass through but prevents the movement of the magnetic particles.

15. The operation method according to claim 1, wherein magnetic field operation in the third step is performed by movement of the magnetic solid from the second liquid layer to the gel-like medium layer so that the predetermined amount or the total amount of the magnetic particles existing in the second liquid layer is moved into the gel-like medium layer.
